(19) United States
(12) Patent Application Publication
(10) Pub. No.: US 2017/0008840 A1
(43) Pub. Date: Jan. 12, 2017

(54) SULFUR-CONTAINING PHENOL DERIVATIVES

(71) Applicant: ARKEMA FRANCE, Colombes (FR)

(72) Inventors: Pascal SAINT-LOUIS-AUGUSTIN, Billere (FR); Jean-Benoit CAZAUX, Billere (FR)

(73) Assignee: Arkema France, Colombes (FR)

(21) Appl. No.: 15/119,533

(22) PCT Filed: Feb. 17, 2015
(86) PCT No.: PCT/FR2015/050385

§ 371 Date: Aug. 17, 2016

(30) Foreign Application Priority Data
Feb. 20, 2014 (FR) FR 1451369

(51) Int. Cl. C07C 323/52; C10M 135/26; C08K 5/375; C09J 11/06; C07C 319/16; C09K 15/14

(52) U.S. Cl. CPC C07C 323/52; C07C 319/16; C09K 15/14; C08K 5/375; C09J 11/06; C10M 135/26

(57) ABSTRACT

The present invention relates to a novel process for preparing sulfur-containing phenolic compounds, and to novel phenol derivatives substituted with at least one radical comprising a sulfur atom. Said sulfur-containing phenolic compounds are of use in numerous industrial fields, particularly the chemical industry, and said compounds are particularly suitable as, for example, antioxidant agents, UV stabilizers, heat stabilizers, and for other uses.

The present invention relates to a novel process for preparing sulfureous phenolic compounds, and novel phenol derivatives substituted with at least one radical comprising a sulfur atom. These sulfureous phenolic compounds find uses in many fields of industry, especially the chemical industry, and are, for example, particularly suitable as antioxidants, UV stabilizers, heat stabilizers and the like.

The literature already provides many examples of phenolic derivatives, in particular of sulfureous phenolic derivatives. However, industry is constantly in search of compounds that are ever more efficient, less toxic, less odorous, more environmentally friendly, easier to prepare, at reduced cost, to mention but the main reasons.

As regards antioxidants, international patent application WO 1997/014 678 discloses compounds comprising phenol groups of formula (A) below:

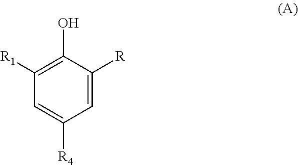

in which R, R₁ and/or R₄ may represent a group of —CH₂—S—(CH₂)ₙ-ester or —CH₂—S—(CH₂)ₙ-amide type, and in which n is equal to 1 or 2.

U.S. Pat. No. 4,759,862 describes the preparation of phenols of formula (B) below, which are used as stabilizers for polymers and oils:

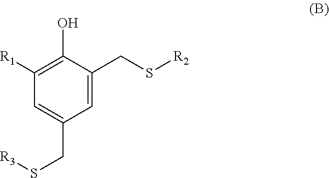

in which formula (B) R₂ and R₃ may each represent a group of —CH₂—S—(CH₂)₁₋₃—W type, in which W represents an ester or amide group.

U.S. Pat. No. 4,857,572 describes the preparation of stabilizers having the structure of substituted phenols of general formula (C) below:

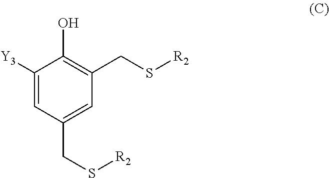

According to this general formula, the substituted phenols may comprise groups of —CH₂—S—CH₂—C(O)OCH₃ type, and/or of C₁-C₁₈ alkyl type, optionally alkyl substituted with —COOR₅.

U.S. Pat. No. 4,091,037 describes the preparation of alkylthiomethylphenols whose general structure (D) is represented below:

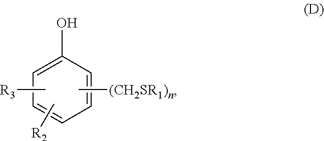

in which general structure (D) the group R₁ cannot comprise an ester group.

U.S. Pat. No. 4,874,885 describes a process for preparing mercaptomethylphenols of general structure (E) below:

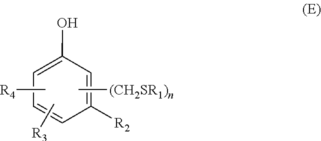

in which the group R₁ may represent a (C₁-C₂₀)-alkyl group or a (C₁-C₄)-alkylene-C(O)OR₅ group.

U.S. Pat. No. 3,227,677 claims the preparation of bis(hydrocarboxy-carbonylalkylthioalkyl)phenols of general formula (F) below:

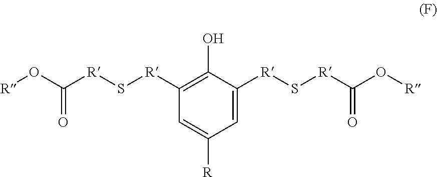

in which R represents hydrogen or a C₁-C₇ alkyl, R′ represents a C₁-C₇ alkylene and R″ is a C₄-C₁₈ alkyl, aryl and/or cycloalkyl.

This general formula shows that the only compounds described are those comprising two groups —R′—S—R′—C(O)OR″, in which R′ represents an alkylene group comprising from 1 to 7 carbon atoms and R″ represents an alkyl, aryl and/or cycloalkyl group of 4 to 18 carbon atoms.

Patent application US 2008/0 081 929 describes a method for preparing thiomethylphenols of structure (G) represented below:

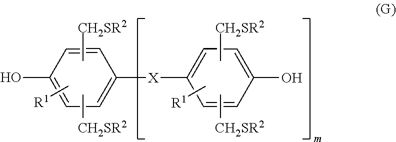

from the corresponding phenolic precursors and the mercaptans of formula R²SH, in which R² represents a linear or branched C₁-C₁₆ alkyl radical, optionally comprising an aryl radical.

Patent DE 198 22 251 discloses compounds of structure (H):

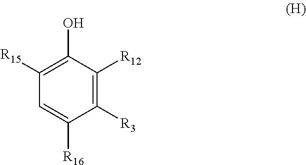

in which at least one of the radicals R₃, R₁₂, R₁₅ and R₁₆ represents a group —CₙH₂ₙ—S—CₘCH₂ₘ—COOR₁₃, in which R₁₃ represents hydrogen or alkyl, n represents 0, 1 or 2 and m represents 1 or 2.

U.S. Pat. No. 6,028,131 describes the preparation of antioxidants corresponding to formula (I) below:

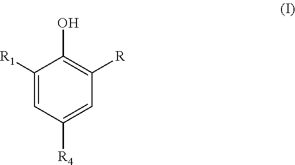

in which R may represent a group —CH₂-A-R₂, in which A represents S or SO and R₂ may be a group —(CH₂)ₘ—C(O)OR₅, in which m is equal to 1 or 2 and R₅ is a C₁-C₁₈ alkyl radical; the other radicals R₁ and R₄ not comprising any sulfur atoms.

A first object of the present invention consists in proposing a novel process for preparing such sulfureous phenolic compounds, said process being simpler to perform, especially industrially, more environmentally friendly, using compounds that are less toxic than those used in the processes for preparing sulfureous phenolic compounds known from the prior art, and more generally using starting materials of bio-sourced origin, in particular starting materials derived from raw materials of plant or animal origin.

One of the advantages of the preparation process according to the invention lies in the fact that the sulfureous phenolic compounds obtained have little or no odour, in particular little or no unpleasant odour, such as may be perceived with certain sulfureous phenolic compounds prepared according to the known preparation processes and which lead to sulfureous phenolic compounds containing traces of nauseating unreacted starting materials, such as mercaptans, in particular certain n-alkyl mercaptans.

The preparation process according to the invention also makes it possible to produce novel sulfureous phenolic compounds that are at least partly prepared from renewable raw materials, and more particularly from fatty acids of plant or animal origin. The present invention thus offers a person skilled in the art novel sulfureous phenolic compounds that are less toxic, more environmentally friendly, and which have little or no odour, in particular little or no unpleasant odour, such as may be perceived with certain known sulfureous phenolic compounds and which comprise traces of nauseating unreacted starting materials, such as mercaptans, in particular certain n-alkyl mercaptans.

Like the known sulfureous phenolic compounds of the prior art, the novel sulfureous phenolic compounds of the invention may, for example, and in a non-limiting manner, be used as antioxidants, UV stabilizers, heat stabilizers, in numerous applications, and in particular in the preparation of plastics, synthetic fibres, elastomers, adhesives, lubricant additives, etc.

Yet other objects will emerge from the description that follows. The abovementioned objects are totally or at least partially achieved, by means of the compounds of the present invention.

Thus, and according to a first aspect, a subject of the present invention is a process for preparing, from raw materials of renewable origin, sulfureous phenolic compounds corresponding to formula (1) below:

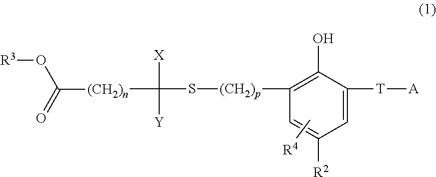

in which:
A represents a radical R¹ or a radical of formula A^{m}:

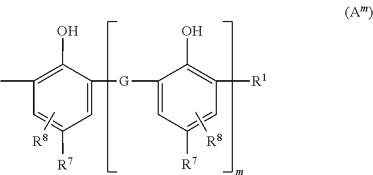

R¹ is chosen from a hydrogen atom, a linear or branched hydrocarbon-based group comprising from 1 to 20 carbon atoms and a group —(CH₂)ₚ—S—C(XY)—(CH₂)ₙ—C(O)OR³;
R² is chosen from a linear or branched hydrocarbon-based group comprising from 1 to 20 carbon atoms and a group —(CH₂)ₚ—S—C(XY)—(CH₂)ₙ—C(O)OR³;
R³ represents a linear or branched hydrocarbon-based group comprising from 1 to 20 carbon atoms;
R⁴, R⁷ and R⁸, which may be identical or different, are chosen, independently of each other, from a hydrogen atom, a linear or branched hydrocarbon-based group comprising from 1 to 20 carbon atoms and a group —(CH₂)ₚ—S—C(XY)—(CH₂)ₙ—C(O)OR³;
G is chosen from —S_{c}—, —(CH₂)ₐ13 , —C(CH₃)₂—, —[S(O)_{b}]_{c}— and —W—, in which W is an aromatic group, optionally substituted with one or more alkyl groups;
T is chosen from a single bond, —Sᵥ—, —(CH₂)ₜ—, —C(CH₃)₂— and —[S(O)ᵤ]ᵥ—;
X and Y, independently of each other, each represent a radical chosen from a hydrogen atom and linear or branched hydrocarbon-based groups, comprising from 1 to 20 carbon atoms, and optionally comprising one or more heteroatoms chosen from oxygen, nitrogen and sulfur;
a and t, which may be identical or different and independently of each other, each represent an integer between 1 and 9 and preferably between 1 and 3, limits inclusive;

b and u, which may be identical or different and independently of each other, each represent an integer equal to 1 or 2;
c and v, which may be identical or different and independently of each other, each represent an integer between 1 and 6, limits inclusive;
m represents 0, or an integer between 1 and 20, limits inclusive;
n represents an integer between 8 and 20, limits inclusive;
p represents an integer between 1 and 10, limits inclusive;
it being understood that at least one of the groups R¹, R² or R⁴ represents a group —(CH₂)ₚ—S—C(XY)—(CH₂)ₙ—C(O)OR³, in which R³, X, Y, n and p are as defined above.

The term “linear or branched hydrocarbon-based group comprising from 1 to 20 carbon atoms” means linear or branched, saturated or unsaturated hydrocarbon-based chains comprising from 1 to 20 carbon atoms, preferably from 1 to 12 carbon atoms and more preferably from 1 to 8 carbon atoms, chosen, for example, from methyl, ethyl, propyl (n- or iso-propyl), butyl (n-, iso- or tert-butyl), pentyl (n-, iso- or neo-pentyl), hexyl, hexenyl, heptyl, heptenyl, octyl, octenyl, nonyl, nonenyl, decyl, decenyl, undecyl, undecenyl, dodecyl and dodecenyl, preferably chosen from methyl, ethyl, n-propyl, isopropyl, n-butyl, isobutyl and tert-butyl.

The preferred compounds of formula (1) are those for which X and Y each represent a hydrogen atom. According to another embodiment, the preferred compounds of formula (1) are those for which X represents a methyl radical, an ethyl radical or a propyl radical and Y represents a hydrogen atom. The preferred compounds of formula (1) are also those for which R⁴ represents a hydrogen atom.

In one embodiment, the preferred compounds of formula (1) are those in which -T- is chosen from a single bond, —Sᵥ—, —(CH₂)ₜ, —C(CH₃)₂—, —[S(O)ᵤ]ᵥ—, in which v represents an integer between 1 and 6, limits inclusive, preferably 1 to 4, t is an integer between 1 and 9, preferably between 1 and 3, limits inclusive, and u is preferably equal to 2.

According to another embodiment, the compounds of formula (1) are those for which m represents 0. The compounds of formula (1) for which m represents 0 (zero) will be identified hereinbelow with the formula (1₀). As a variant, the compounds of formula (1) for which m is other than 0 are identified by the formula (1ₘ). When m is other than 0, preference is given to the compounds of formula (1ₘ) for which m represents an integer preferably between 1 and 10, limits inclusive, more preferably m is equal to 1, 2, 3, 4, 5 or 6. All the compounds of formula (1ₘ) with those of formula (1₀) form the set of compounds of formula (1).

The compounds of formula (1₀) for which p is strictly greater than 1 and less than or equal to 10, with the compounds of formula (1ₘ) for which p represents an integer between 1 and 10, limits inclusive, form the compounds of general formula (1″). These compounds of general formula (1″) are novel and, in this respect, form another subject of the present invention, as indicated later.

According to yet another embodiment, the preferred compounds of formula (1) are those for which n represents 8, 9, 10, 11 or 12, more preferably 8 or 9. The preferred compounds of formula (1) are also those for which p represents 1, 2, 3 or 4, preferably 1 or 2, and, entirely preferably, p is equal to 1.

Another preferred embodiment of the present invention concerns the process for preparing the compounds of formula (1) comprising at least two, preferably at least three and more preferably at least 4 groups —(CH₂)ₚ—S—C(XY)—(CH₂)ₙ—C(O)OR³, in which R³, X, Y, n and p are as defined previously. In the compounds of formula (1ₘ), it should be understood that the radicals R⁷, on the one hand, and R⁸, on the other hand, may be identical or different, and are preferably identical.

The compounds of formula (1) that are most particularly preferred are those having at least one, at least two, at least three, at least four or even all of the following characteristics:
R¹ is chosen from a hydrogen atom, a linear or branched hydrocarbon-based group comprising from 1 to 12 carbon atoms and a group —(CH₂)ₚ—S—C(XY)—(CH₂)ₙ—C(O)OR³;
R² is chosen from a linear or branched hydrocarbon-based group comprising from 1 to 12 carbon atoms and a group —(CH₂)ₚ—S—C(XY)—(CH₂)ₙ—C(O)OR³;
R³ represents a linear or branched hydrocarbon-based group comprising from 1 to 6 carbon atoms;
R⁴ is chosen from a hydrogen atom and a linear or branched hydrocarbon-based group comprising from 1 to 6 carbon atoms;
T is chosen from a single bond, —Sᵥ—, —(CH₂)ₜ— and —C(CH₃)₂—;
X and Y, independently of each other, each represent a radical chosen from a hydrogen atom and linear or branched alkyl radicals comprising from 1 to 6 carbon atoms;
t represents an integer between 1 and 3, limits inclusive;
v represents an integer between 1 and 6, limits inclusive;
m represents 0, or an integer between 1 and 10, limits inclusive;
n represents an integer between 8 and 20, limits inclusive;
p represents 1 or 2, preferably p is equal to 1;
it being understood that at least one of the groups R¹ or R² represents a group —(CH₂)ₚ—S—C(XY)—(CH₂)ₙ—C(O)OR³, in which R³, X, Y, n and p are as defined above.

The compounds of formula (1) that are most particularly preferred are those having at least one, preferably at least two, preferably at least three, preferably at least four and preferably all of the following characteristics:
R¹ is chosen from a hydrogen atom and a linear or branched hydrocarbon-based group comprising from 1 to 6 carbon atoms;
R² represents a group —(CH₂)ₚ—S—C(XY)—(CH₂)ₙ—C(O)OR³;
R³ represents methyl or ethyl;
R⁴ represents a hydrogen atom;
T is chosen from a single bond, —Sᵥ—, —(CH₂)ₜ— and —C(CH₃)₂—;
X represents a radical chosen from a hydrogen atom and methyl, ethyl, propyl and butyl radicals;
Y represents a hydrogen atom;
t represents an integer between 1 and 3, limits inclusive;
v represents an integer between 1 and 6, limits inclusive;
m represents 0, or an integer between 1 and 6, limits inclusive;
n represents an integer between 8 and 12, limits inclusive; and
p is equal to 1.

Thus, the present invention relates to a process for preparing the compounds of formula (1) from raw materials of renewable origin, and more particularly from fatty acids of plant or animal origin. More particularly, the present invention relates to a process for preparing a compound of formula (1) as defined above, said process comprising at least steps a) to c) below:
a) reaction of a mercapto alkoxide of formula (2):

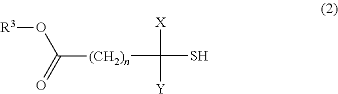

in which R³, X, Y and n are as defined previously, with an amine compound of formula (3):

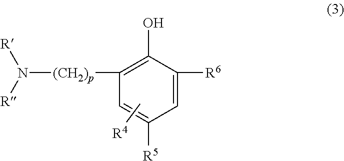

in which R⁴ and p are as defined previously and
R′ and R″, which may be identical or different, are chosen, independently of each other, from a hydrogen atom, a linear or branched alkyl radical comprising from 1 to 6 carbon atoms, or form, together with the nitrogen atom that bears them, a heterocycle,
R⁵ represents a linear or branched hydrocarbon-based group comprising from 1 to 20 carbon atoms or a group —(CH₂)ₚ—NR′R″, and
R⁶ is chosen from a hydrogen atom, a linear or branched hydrocarbon-based group comprising from 1 to 20 carbon atoms and a group —(CH₂)ₚ—NR′R″,
b) optionally, reaction of the compound obtained in step a) with a compound of formula (4):

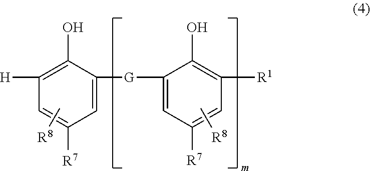

in which R¹, R⁷, R⁸, m and G are as defined previously, via an aromatic electrophilic substitution reaction, according to the techniques well known to those skilled in the art, and
c) extraction and then optional purification of the compound of formula (1).

Among the heterocycles formed by R′, R″ with the nitrogen atom that bears them, mention may be made, in a non-limiting manner and purely as examples, of saturated or unsaturated 4-, 5- or 6-membered heterocycles, which may contain one or more other heteroatoms preferably chosen from oxygen, nitrogen and sulfur. Such heteroatoms are well known to those skilled in the art, are described, for example, in patent application US 2008/0 081 929 and are preferably piperidine, pyrrolidine and piperazine.

The condensation reaction of the compound of formula (2) with the compound of formula (3) may be performed according to any method known to those skilled in the art. This reaction may be performed in solvent medium or without solvent, optionally in the presence of a catalyst, at a temperature typically, but not exclusively, between 90° C. and 150° C., preferably at atmospheric pressure, for a time ranging between one hour and a few hours, for example between 2 hours and 36 hours, depending on the substrates under consideration, the temperature and the pressure in the reaction medium.

The solvents that may be used in the process of the present invention are of any type known to those skilled in the art and especially organic, aqueous and aqueous-organic solvents. Typical examples of solvents that may be used in the process of the invention comprise water, alcohols (in particular methanol or ethanol), glycols, and also mixtures of two or more thereof in all proportions.

The compound of formula (4) is subjected to an aromatic electrophilic substitution reaction in the presence of the compound obtained in step a), optionally in the presence of a reagent that is a precursor of the group T and optionally of a catalyst of Lewis acid type, according to techniques that are well known to those skilled in the art and described, for example, in Advanced Organic Chemistry, M. B. Smith & J. March, 5th edition, 2001, chapter 11, pp. 675 et seq.

The compounds of formula (4) may typically be a phenol (in the case of the compounds of formula (4) in which m represents 0) or a phenolic resin (in the case of the compounds of formula (4) in which m is other than 0). The compounds of formula (4) are known and commercially available or readily prepared from procedures that are known and available in the scientific literature, the patent literature, Chemical Abstracts or the Internet.

Non-limiting examples of phenolic resins include, as non-limiting illustrations of the invention:
phenol/aldehyde resins, of novolac type, derived, for example, from the reaction of para-alkylphenol with paraformaldehyde in the presence of a Lewis acid; in this case, the reaction with the synthon obtained in step (a) is preferably performed in the presence of paraformaldehyde and of a Lewis acid;
sulfureous phenolic resins, of Vultac® type, which may be derived from the reaction of para-alkylphenol with sulfur chloride (and optionally additional sulfur) and also optionally a Lewis acid; in this case, the reaction with the synthon obtained in step (a) is preferably performed in the presence of sulfur chloride or sulfur dichloride, and optionally a Lewis acid, as described, for example, in document WO 2005/037 910;
phenolic resins with gem-dimethyl bridges derived from the oligomerization reaction of ortho-isopropenyl-para-alkylphenol optionally in the presence of a Lewis acid; in this case, the reaction with the synthon obtained in step (a) is preferably performed in the presence of a Lewis acid.

The compounds of formulae (2), (3) and (4) are known and commercially available or are readily prepared from procedures that are known and available in patents, the scientific literature, Chemical Abstracts, or on the Internet.

In an entirely advantageous manner, the compounds of formula (2) may be obtained from plant or animal oils or fats, and/or from natural fatty acids, according to processes known to those skilled in the art.

By way of example, the compounds of formula (2) may be obtained according to the processes described in patents FR 2 424 907, FR 2 603 889 and in patent application US 2012/0 232 297.

According to a particularly preferred embodiment, the compounds of formula (2) are obtained from plant or animal oils or fats, which are predominantly in the form of triglycerides. These triglycerides are subjected (step a1) to a basic or acidic catalytic transesterification reaction, in the presence of an alcohol, preferably an aliphatic alcohol, typically methanol or ethanol, to give the corresponding fatty acid esters, with removal of glycerol.

The fatty acid esters generally and usually comprise one or more double bonds, then subjected (step a) to a sulfhydration reaction, so as to give the mercapto ester of formula (2).

As a variant, one or more metathesis reactions may be performed on the triglycerides and/or the fatty acid esters, in order to modify or isomerize the double bond(s) present on the fatty chains, as described, for example, in US 2012/0 232 297.

The plant or animal oils or fats, referred to hereinbelow as “natural oils”, which are used in step a1) may be of any type known to those skilled in the art, and especially fatty acid triesters of glycerol (which may also contain mono- and di-glycerides) and which are found in abundance in nature, for example in oil-yielding plants and animal fats, to mention but the most important sources of triglycerides, in particular of unsaturated triglycerides, i.e. those comprising a carbon-carbon double bond.

The fatty acid triglycerides may also contain a more or less large amount of free fatty acids. When this amount is relatively large, typically greater than about 5% by weight, it may be advantageous to perform a pretreatment of the triglycerides consisting of a first esterification of said free fatty acids in the presence of an alcohol and of an acid catalyst, such as sulfuric acid or methanesulfonic acid, as described, for example, in patent FR 2 929 621. The free fatty acids contained beforehand in the triglycerides are thus converted into esters.

The free fatty acids present in the starting triglycerides may also be found in lower amounts, typically between 0.1% by weight and 5% by weight, and, in these cases, a basic wash may be sufficient to remove them in the form of basic salts.

Step a1) is a step of transesterification of the natural oil (after optional pretreatment of the free fatty acids, as indicated above) allowing the glycerol to be removed.

The unsaturated glycerides that may be used originate essentially from animal or plant, preferably plant, oils or fats, among which mention may be made, as non-limiting indications, of soybean oil, sunflower oil, linseed oil, rapeseed oil, castor oil, palm oil, palm kernel oil, coconut oil, jatropha oil, cotton seed oil, groundnut oil, olive oil, vernonia oil, cuphea oil, hevea oil, lunaria oil, safflower oil, camellina oil, Calophyllum inophyllum oil, Pongamia pinnata oil, beef tallow, cooking oil or grease, but may also be hydraulic or lubricant oils.

The transesterification reaction of the triglycerides is generally performed in basic medium, in the presence of an alcohol, generally a monoalcohol, comprising from 1 to 20 carbon atoms, preferably from 1 to 12 carbon atoms, more preferably from 1 to 6 carbon atoms, most particularly preferably from 1 to 4 carbon atoms and entirely preferably a low molecular weight alcohol, for example methanol or ethanol, methanol being most commonly used for mainly economic reasons. The transesterification reaction allows the production of fatty acid esters, for example of methyl esters when the alcohol used is methanol.

The basic compounds that may be used for the transesterification reaction may be of any type known to those skilled in the art, and may be chosen in particular from alkali metal and alkaline-earth metal oxides, hydrides, hydroxides, carbonates, hydrogen carbonates, acetates and other alkoxides, the alkoxides originating from alcohols preferably comprising from 1 to 5 carbon atoms. Among these basic compounds, sodium hydroxide, potassium hydroxide, sodium alkoxides and potassium alkoxides are preferred. In an entirely preferred manner, the basic compounds are chosen from sodium hydroxide, potassium hydroxide, sodium methoxide and potassium methoxide, the latter two alkoxides being most particularly preferred.

The transesterification reaction may also be performed in acidic medium as indicated in international patent application WO 2011/018 228. In this case, use may be made, for example, of methanesulfonic acid in aqueous solution sold by the company Arkema, for example an aqueous solution of methanesulfonic acid at 70% by weight in water, or anhydrous methanesulfonic acid or AMSA. Transesterification in acidic medium also has the advantage of simultaneously allowing the esterification of the free fatty acids that may be present in the fatty acid triglycerides.

After the transesterification reaction, the fatty acid esters are present in the reaction medium with glycerol. This reaction medium may comprise more or less large amounts of water depending on the conditions under which the transesterification reaction was performed. Glycerol and the water, if any, are not soluble in fatty acid esters and are separated therefrom by decantation or by any other means that allows phase separation.

As indicated previously, the unsaturated esters that are precursors of the compounds of formula (2) may also be obtained by cross-metathesis from other unsaturated esters, or even from glycerides (the latter will then be subjected to a transesterification step with an alcohol as indicated above), for instance those defined previously. Metathesis reactions are well known to those skilled in the art and usually involve an intermolecular reaction between two compounds each bearing at least one double bond, as described, for example, in patent applications WO 2009/047 444 and US 2012/0 232 297.

These metathesis reactions are advantageously performed starting with unsaturated methyl esters and, for example, in a non-limiting manner, those chosen from methyl palmitoleate, methyl arachidonate, alone or as mixtures of two or more thereof in all proportions. According to this embodiment, dimethyl 9-octadecen-1,18-dioate may, for example, be readily obtained by metathesis of methyl oleate and/or of methyl palmitoleate.

The sources of unsaturated esters are thus very numerous and varied, and non-limiting indicative examples of methyl esters bearing an unsaturation which may be sulfhydrated comprise, in a non-limiting indicative manner, methyl hexenoates, methyl decenoates, methyl undecenoates, methyl dodecenoates, methyl oleate, methyl linoleate, methyl myristoleate, methyl palmitoleate, methyl linoleate, methyl linolenate, methyl arachidonate, methyl ricinoleate, dimethyl 9-octadecen-1,18-dioate, and also mixtures of two or more thereof in all proportions.

Preferably, the unsaturated esters are chosen from methyl decenoates and methyl undecenoates, more preferably from methyl decen-9-oate and methyl undecen-10-oate.

According to another variant, the unsaturated esters that are precursors of the compounds of formula (2) may also be obtained from the corresponding acids, which are subjected to an esterification reaction, in the presence of an alcohol, for example methanol, according to standard esterification techniques that are well known to those skilled in the art.

Non-limiting indicative examples of acids that are precursors of the unsaturated esters comprise, in a non-limiting manner, hexenoic, decenoic, undecenoic, dodecenoic, oleic, linoleic, myristic, palmitic, linoleic, linolenic, arachidonic and ricinoleic acids, di-acids and tri-acids that may be obtained by cross-metathesis according to the standard methods of synthesis by metathesis, as indicated above, for example 9-octadecen-1,18-dioic acid. Preferably, said acids are chosen from decenoic and undecenoic acids and mixtures of two or more thereof in all proportions, more preferably from 9-decenoic acid and 10-undecenoic acid.

The compounds of formula (2) may be readily prepared from the unsaturated esters described above by a sulfhydration reaction (step a2) according to the techniques known to those skilled in the art. The term “sulfhydration reaction” means the introduction of an —SH group onto an unsaturation as illustrated in the scheme below:

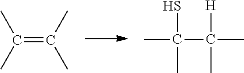

The carbon-carbon double bond present in the unsaturated ester may thus be sulfhydrated in one or two steps, according to a standard radical addition reaction via the action of hydrogen sulfide (as described, for example, in FR 2 424 907) or a hydrogen sulfide precursor, for example thioacetic acid (as described, for example, in U.S. Pat. No. 4,701,492), a tertiary mercaptan, for example tert-butyl mercaptan (as described, for example, in FR 2 603 889), or via a catalytic addition of hydrogen sulfide (as described, for example, in U.S. Pat. No. 4,102,931).

Thus, the sulfhydrating agent used for the sulfhydration of the unsaturated ester to a compound of formula (2) may be of any type known to those skilled in the art and may be chosen, for example, from hydrogen sulfide, thioacetic acid (TAA), and other compounds known to those skilled in the art and usually used in sulfhydration reactions of organic compounds.

This sulfhydration reaction is advantageously performed in the presence of a homogeneous or heterogeneous acid catalyst and/or under the ultraviolet (UV) light irradiation, either by direct photolysis at wavelengths of between 180 nm and 300 nm, or in the presence of photoinitiators. According to a preferred embodiment, the sulfhydration reaction is performed without catalyst, and under UV irradiation.

This sulfhydration reaction may be performed in the presence or absence of solvent, preferably in the presence of one or more solvents, which may be advantageously chosen for their transparency to UV light depending on the wavelength used and the ease of separating them from the reaction medium. Such solvents may be chosen, for example, from light alkanes (1 to 6 carbon atoms), ethylene glycol ethers, aromatic hydrocarbons, aliphatic hydrocarbons, and the like, and also mixtures of two or more thereof in all proportions.

As a variant, the sulfhydration reaction may be performed in the presence of one or more, preferably one, compound(s) capable of forming free radicals. Such compounds are known to those skilled in the art and may be chosen, for example, from peroxides, and, in a non-limiting indicative manner, from hydrogen peroxide, sodium peroxide, potassium peroxide, tert-alkyl (for example tert-butyl) hydroperoxides, tert-alkyl peroxides, tert-alkyl peresters, cumene hydroperoxide, azobisisobutyronitrile, and the like, and mixtures of two or more thereof in all proportions.

When the sulfhydration reaction described above is performed via the action of thioacetic acid in the presence of a free radical initiator and/or by irradiation with UV light, as described previously, this reaction is followed by a methanolysis reaction in acidic medium, for releasing the desired mercaptan of formula (2) and also methyl acetate removed (for example, and in a non-limiting manner) continuously from the medium by azeotropic distillation. This methanolysis reaction is well known and may be performed according to any standard technique.

After the sulfhydration step (step a2), the mercapto esters of formula (2) may be obtained in the form of mixtures of isomers (primary, secondary and/or tertiary mercaptans) which may then be separated and optionally purified according to standard separation and/or purification techniques, for example by distillation, under atmospheric pressure or under reduced pressure depending on the nature of the mercaptan of interest to be recovered.

As a variant, and according to yet another embodiment, the unsaturated esters that are precursors of the compounds of formula (2) may be obtained from unsaturated fatty esters, or even from glycerides, for instance those defined previously, the latter then being subjected beforehand to a step of transesterification with an alcohol as indicated above. These unsaturated fatty esters may then be treated via pyrolysis according to techniques that are well known to those skilled in the art. This treatment by pyrolysis is particularly suited to the treatment of methyl ricinoleate, for which pyrolysis makes it possible to selectively obtain methyl 10-undecenoate.

The unsaturated esters resulting from one or more of these treatments (transesterification, metathesis, pyrolysis and the like), and after separation of the reaction products (especially unsaturated diesters, unsaturated monoesters, alkenes and the like), are then subjected to a sulfhydration reaction as described previously to obtain the compounds of formula (2).

Examples of compounds of formula (2) that are particularly preferred for the synthesis of the compounds of formula (1) according to the present invention are, in a non-limiting manner, methyl mercaptodecanoate and methyl mercaptoundecanoate.

The process for preparing the compounds of formula (1) according to the invention also comprises the reaction of at least one compound of formula (2) described above with a phenolic derivative of formula (3), as defined above.

The phenolic derivatives of formula (3) are amine phenolic derivatives that are known and readily commercially available or readily prepared from procedures known in patents, the scientific literature, Chemical Abstracts or the Internet. These compounds may also be non-isolated synthetic intermediates described in the literature (US 2008/081 929).

Each amine group of the compound of formula (3) reacts with a mercapto ester of formula (2) under the known conditions described in the literature, for example in documents US 2008/081 929 and U.S. Pat. No. 4 857 572.

Examples of compounds of formula (3) comprise, as non-limiting examples, 2-(N,N-dimethylaminoethyl)phenol (CAS No. 94-54-2), 2,4-bis[(N,N-dimethylamino)methyl]-6-methylphenol (CAS No. 5424-54-4) and 2,4,6-tris(N,N-dimethylaminomethyl)phenol (CAS No. 90-72-2).

The compounds obtained after reaction of a compound of formula (2) with a compound of formula (3) correspond to the compounds of formula (1_{A}) in which T represents a bond and A represents R¹.

The compounds of formula (1), other than the compounds of formula (1_{A}), i.e. the compounds for which T does not represent a bond and/or A represents the radical (A^{m}), may be readily obtained by reacting a reagent bearing the group T and/or a compound of formula (4) defined previously, as indicated above, according to methods that are well known to those skilled in the art.

As non-limiting examples:
the compounds of formula (1) for which T represents S may be obtained from a compound of formula (1_{A}) as defined previously with R¹ representing a hydrogen atom, and a compound of formula (4), in the presence of sulfur dichloride (SCl₂) or sulfur chloride (S₂Cl₂), in the presence of a solvent, at a temperature of between −10° C. and 40° C., for a time generally ranging between 30 minutes and 3 hours;
the compounds of formula (1) for which T represents S_{c}, in which c represents 2, 3, 4, 5 or 6, may be obtained from a compound of formula (1_{A}) as defined previously with R¹ representing a hydrogen atom, and a compound of formula (4), in the presence of sulfur chloride (S₂Cl₂), optionally sulfur (S₈) and optionally an organic solvent, at a temperature between 50° C. and 160° C., for a time generally between 30 minutes and 3 hours;
the compounds of formula (1) for which T represents —(CH₂)— may be obtained from a compound of formula (1_{A}), as defined previously with R¹ representing a hydrogen atom, and a compound of formula (4), in the presence of formaldehyde (CH₂O), at a temperature generally between 60° C. and 150° C.;
the compounds of formula (1) for which T represents —C(CH₃)₂— may be obtained from a compound of formula (1_{A}) for which R¹ represents an isopropenyl group and a compound of formula (4), in the presence of a Lewis acid, such as triethylaluminium, and of a solvent, and in a temperature range from 60° C. to 150° C., for a time which may range from 30 minutes to 6 hours, and
the compounds of formula (1) for which T represents —[S(O)_{b}]_{c}— may be obtained from a compound of formula (1_{A}) for which T represents S_{c}, in which c represents 2, 3, 4, 5 or 6, in the presence of an oxidizing agent (such as hydrogen peroxide) and of an organic solvent, at a temperature between 0° C. and 40° C. for a time of between 1 hour and 48 hours.

The process according to the present invention for preparing the compounds of formula (1), as defined previously, from a triglyceride, preferably of natural origin, thus comprises at least the following steps:
a0) provision of at least one triglyceride;
a1) transesterification of said at least one triglyceride, in the presence of an alcohol, and removal of the glycerol formed, to obtain an unsaturated ester;
a1′) optional treatment by metathesis or pyrolysis of said unsaturated ester;
a2) sulfhydration of the unsaturated ester from step a1) or a1′) to obtain the mercapto ester of formula (2), as defined previously;
a) reaction of the mercapto ester of formula (2) as defined previously with an amine phenolic compound of formula (3) as defined previously, to obtain the compounds of formula (1_{A}) as defined previously;
b) optionally, reaction of the compound obtained in step a) with a compound of formula (4), optionally in the presence of a reagent bearing the group T; c) extraction and then optional purification of the compounds obtained in step b).

As indicated previously, the compounds of formula (1₀) for which p is strictly greater than 1 and less than or equal to 10, with the compounds of formula (1ₘ) for which p represents an integer between 1 and 10, limits inclusive, are novel and, in this respect, form another subject of the present invention.

Thus, another subject of the present invention relates to the compounds of formula (1″):

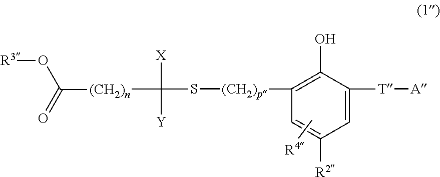

in which:
A″ represents a radical R¹″ or a radical of formula A^{m}″:

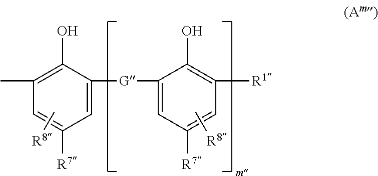

R¹″ is chosen from a hydrogen atom, a linear or branched hydrocarbon-based group comprising from 1 to 20 carbon atoms and a group —(CH₂)_{p″}—S—C(X″Y″)—(CH₂)_{n″}—C(O)OR³″;
R²″ is chosen from a linear or branched hydrocarbon-based group comprising from 1 to 20 carbon atoms and a group —(CH₂)_{p″}—S—C(X″Y″)—(CH₂)_{n″}—C(O)OR³″;
R³″ represents a linear or branched hydrocarbon-based group comprising from 1 to 20 carbon atoms;
R⁴″, R⁷″ and R⁸″, which may be identical or different, are chosen, independently of each other, from a hydrogen atom, a linear or branched hydrocarbon-based group comprising from 1 to 20 carbon atoms and a group —(CH₂)_{p″}—S—C(X″Y″)—(CH₂)_{n″}—C(O)OR³″;
G″ is chosen from —S_{c″}—, —(CH₂)_{a″}—, —C(CH₃)₂—, —[S(O)_{b″}]_{c″}— and —W″—, in which W″ is an aromatic group, optionally substituted with one or more alkyl groups;
T″ is chosen from a single bond, —S_{v″}—, —(CH₂)_{t″}—, —C(CH₃)₂—, and —[S(O)_{u″}]_{v″}—;
X″ and Y″, independently of each other, each represent a radical chosen from a hydrogen atom and linear or branched hydrocarbon-based groups, comprising from 1 to 20 carbon atoms, and optionally comprising one or more heteroatoms chosen from oxygen, nitrogen and sulfur;
a″ and t″, which may be identical or different and independently of each other, each represent an integer between 1 and 9 and preferably between 1 and 3, limits inclusive;
b″ and u″, which may be identical or different and independently of each other, each represent an integer equal to 1 or 2;
c″ and v″, which may be identical or different and independently of each other, each represent an integer between 1 and 6, limits inclusive;
m″ represents 0, or an integer between 1 and 20, limits inclusive;
n″ represents an integer between 8 and 20, limits inclusive;
p″ represents an integer between 1 and 10, limits inclusive;
it being understood that at least one of the groups R¹″, R²″ or R⁴″ represents a group —(CH₂)_{p″}—S—C(X″Y″)—(CH₂)ₙ—C(O)OR³″, in which R³″, X″, Y″, n″ and p″ are as defined above, and
it being understood that when p″=1, then A″ represents a radical of formula A^{m}″.

The set of compounds of formula (1″) is included in the set of compounds of formula (1). Thus, the compounds of formula (1″) may be prepared according to the procedure described for obtaining the compounds of formula (1). Similarly, and unless otherwise indicated, the definitions and preferences given for the various substituents on the compounds of formula (1) also apply to the substituents on the compounds of formula (1″).

In particular, the preferred compounds of formula (1″) are those for which X″ and Y″ each represent a hydrogen atom. The preferred compounds of formula (1″) are also those for which X″ represents a methyl radical, an ethyl radical or a propyl radical and Y″ represents a hydrogen atom. The preferred compounds of formula (1″) are also those for which R⁴″ represents a hydrogen atom.

In one embodiment, the preferred compounds of formula (1″) are those in which -T″- is chosen from a single bond, —S_{v″}—, —(CH₂)_{t″}—, —C(CH₃)₂—, —[S(O)_{u″}]_{t″}—, in which v″ represents an integer between 1 and 6, limits inclusive, preferably 1 to 4, t″ is preferably an integer between 1 and 9 and preferably between 1 and 3, limits inclusive, u″ is preferably equal to 2.

The preferred compounds of formula (1″) are also those for which m″ represents an integer equal to 1, 2, 3, 4, 5 or 6 and p″ represents an integer equal to 1, 2, 3 or 4.

According to yet another embodiment, the preferred compounds of formula (1″) are those for which n″ represents 8, 9, 10, 11 or 12, more preferably 8 or 9. The preferred compounds of formula (1″) are also those for which m″ is equal to 1, 2, 3 or 4, and p″ represents 2, 3 or 4, preferably 2.

Another preferred embodiment of the present invention collates the compounds of formula (1″) comprising 2, 3 or 4, preferably 2 or 3, preferably 2 groups —(CH₂)_{p″}—S—C(X″Y″)—(CH₂)_{n″}—C(O)OR³″, in which R³″, X″, Y″, n″ and p″ are as defined previously. In the compounds of formula (1″), it should be understood that the radicals R²″ may be identical or different, and are preferably identical.

The compounds of formula (1″) that are most particularly preferred are those having at least one, at least two, at least three, at least four, or even all of the following characteristics:
R¹″ is chosen from a hydrogen atom, a linear or branched hydrocarbon-based group comprising from 1 to 12 carbon atoms and a group —(CH₂)_{p″}—S—C(X″Y″)—(CH₂)_{n″}—C(O)OR³″;
R²″ is chosen from a linear or branched hydrocarbon-based group comprising from 1 to 12 carbon atoms and a group —(CH₂)_{p″}—S—C(X″Y″)—(CH₂)_{n″}—C(O)OR³″; p0 R³″ represents a linear or branched hydrocarbon-based group comprising from 1 to 6 carbon atoms;
R⁴″ is chosen from a hydrogen atom and a linear or branched hydrocarbon-based group comprising from 1 to 6 carbon atoms;
T″ is chosen from a single bond, —S_{v″}—, —(CH₂)_{t″}— and —C(CH₃)₂—;
X″ and Y″, independently of each other, each represent a radical chosen from a hydrogen atom and linear or branched alkyl radicals comprising from 1 to 6 carbon atoms;
t″ represents an integer between 1 and 3, limits inclusive;
v″ represents an integer between 1 and 6, limits inclusive;
m″ represents 0, or an integer between 1 and 10, limits inclusive;
n″ represents an integer between 8 and 20, limits inclusive;
p″ represents 2;
it being understood that at least one of the groups R^{1″} or R²″ represents a group —(CH₂)_{p″}—S—C(X″Y″)—(CH₂)_{n″}—C(O)OR³″, in which R³″, X″, Y″, n″ and p″ are as defined above,
   and more preferably those having at least one, at least two, at least three, at least four, or even all of the following characteristics:
R¹″ is chosen from a hydrogen atom and a linear or branched hydrocarbon-based group comprising from 1 to 6 carbon atoms;
R²″ represents a group —(CH₂)_{p″}—S—C(X″Y″)—(CH₂)_{n″}—C(O)OR³″;
R³″ represents methyl or ethyl;
R⁴″ represents a hydrogen atom;
T″ is chosen from a single bond, —S_{v″}—, —(CH₂)_{t″}— and —C(CH₃)₂—;
X″ represents a radical chosen from a hydrogen atom and methyl, ethyl, propyl and butyl radicals;
Y″ represents a hydrogen atom;
t″ represents an integer between 1 and 3, limits inclusive;
v″ represents an integer between 1 and 6, limits inclusive;
m″ represents 0, or an integer between 1 and 6, limits inclusive;
n″ represents an integer between 8 and 12, limits inclusive; and
p″ is equal to 1.

By means of the present invention, it is now possible to propose novel sulfureous phenolic compounds which are less toxic and more environmentally friendly, and which have little or no odour, in particular little or no unpleasant odour, such as that which may be perceived with certain known sulfureous phenolic compounds and which comprise traces of nauseating unreacted starting materials, such as mercaptans, in particular certain n-alkyl mercaptans.

The phenolic compounds according to the present invention have good properties as antioxidants, and particularly improved properties when compared with the antioxidants of the family of phenolic compounds known from the prior art, which allows their use as antioxidants, UV stabilizers, heat stabilizers, and the like.

Thus, and according to yet another aspect, the present invention relates to the use of at least one sulfureous phenolic compound of formula (1″) as defined above, as antioxidant, UV stabilizer or heat stabilizer, in numerous applications, and particularly in the preparation of plastics, synthetic fibres, elastomers, adhesives, additives, lubricants, etc.

The examples that follow illustrate the invention without limiting it.

### EXAMPLE 1

### Preparation of 2-(10-methyl(ethylthio)decanoate)phenol from Soybean Oil

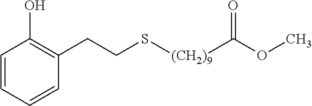

Soybean oil is treated with aluminium so as to reduce its peroxide content to less than 10 milliequivalents per kg of soybean oil. The soybean oil is then degassed by sparging with nitrogen for 30 minutes. The treated soybean oil is then stored in a container under a nitrogen atmosphere until the time of use.

250 ml of deperoxidized and degassed (as previously) soybean oil are placed in a 500 ml stainless-steel autoclave, equipped with a magnetic stirring device, a heating device, a gas introduction valve and a relief valve.

A sealed glass tube containing 35 mg of (tricyclohexylphosphine)(benzylidene)ruthenium chloride dissolved in 5 ml of toluene is placed in the autoclave. The autoclave is closed and then placed under an inert atmosphere of nitrogen via 3 cycles of flushing under vacuum/placing under a slight pressure of nitrogen (550 kPa). The system is then flushed under vacuum a final time, followed by introduction of ethylene into the medium, while maintaining a pressure of 2.7 MPa, which will be maintained throughout the test.

Stirring is started to break the glass tube containing the catalyst, and the temperature of the medium is then raised to 30° C. and maintained for 10 hours.

At the end of the test, the autoclave is cooled and returned to atmospheric pressure, and the contents are purified by passage through alumina to remove the (tricyclohexylphosphine)(benzylidene)ruthenium chloride. The reaction medium is then purified by distillation to separate the side products from the glyceryl tris(9-decenoate). The expected product is obtained in a yield of greater than 70%.

A step of methanolysis of the products is performed on the glyceryl tris(9-decenoate) in order to recover the glycerol, on the one hand, and methyl 9-decenoate, on the other hand.

The methyl 9-decenoate thus obtained (156 g) is placed in a photochemical reactor comprising a reaction loop, with 100 g of pentane and 60 molar equivalents of liquefied hydrogen sulfide (1806 g condensed at 20° C. under a pressure of 17.5 bar, i.e. 1.75 MPa). The mixture is recirculated at a rate of 601/hour in the reaction loop, in which it is subjected to UV radiation (wavelength: 254 nm, power: 12 watts) for 3 hours at a temperature of 38° C. and at a pressure of 23 bar (2.3 MPa).

The excess hydrogen sulfide is then flushed out towards a thermal oxidizer by decompression of the medium, followed by stripping with nitrogen. The mixture is then distilled so as to remove the solvent and the sulfides formed, at a temperature of 130° C., under a pressure of 5 mbar (500 Pa). The methyl 9-mercaptodecanoate is obtained in a purity of greater than 98.5%.

115.7 g (0.7 mol) of 2-(dimethylaminoethyl)phenol and 152.7 g of methyl 10-mercaptodecanoate (0.7 mol) are mixed in a jacketed reactor equipped with a stirring system and a condenser for a time of 36 hours at 150° C. The dimethylamine is removed continuously from the medium by gentle stripping with nitrogen.

The crude reaction product is removed from the medium and washed with water, and the organic phase is distilled under reduced pressure to give the expected 2-(10-methyl(ethylthio)decanoate)phenol.

1. A process for preparing, from raw materials of renewable origin, sulfureous phenolic compounds corresponding to formula (1) below: 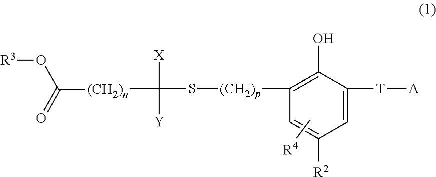
in which:
A represents a radical R¹ or a radical of formula A^{m}: 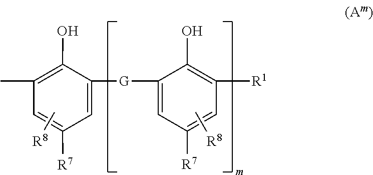
R¹ is chosen from a hydrogen atom, a linear or branched hydrocarbon-based group comprising from 1 to 20 carbon atoms and a group —(CH₂)ₚ—S—C(XY)—(CH₂)ₙ—C(O)OR³;
R² is chosen from a linear or branched hydrocarbon-based group comprising from 1 to 20 carbon atoms and a group —(CH₂)ₚ—S—C(XY)—(CH₂)ₙ—C(O)OR³;
R³ represents a linear or branched hydrocarbon-based group comprising from 1 to 20 carbon atoms;
R⁴, R⁷ and R⁸, which may be identical or different, are chosen, independently of each other, from a hydrogen atom, a linear or branched hydrocarbon-based group comprising from 1 to 20 carbon atoms and a group —(CH₂)ₚ—S—C(XY)—(CH₂)ₙ—C(O)OR³;
G is chosen from —S_{c}—, —(CH₂)ₐ, —C(CH₃)₂—, —[S(O)_{b}]_{c}— and —W—, in which W is an aromatic group, optionally substituted with one or more alkyl groups;
T is chosen from a single bond, —Sᵥ—, —(CH₂)ₜ—, —C(CH₃)₂—, and —[S(O)ᵤ]ᵥ—;
X and Y, independently of each other, each represent a radical chosen from a hydrogen atom and linear or branched hydrocarbon-based groups, comprising from 1 to 20 carbon atoms, and optionally comprising one or more heteroatoms chosen from oxygen, nitrogen and sulfur;
a and t, which may be identical or different and independently of each other, each represent an integer between 1 and 9 and preferably between 1 and 3, limits inclusive;
b and u, which may be identical or different and independently of each other, each represent an integer equal to 1 or 2;
c and v, which may be identical or different and independently of each other, each represent an integer between 1 and 6, limits inclusive;
m represents 0, or an integer between 1 and 20, limits inclusive;
n represents an integer between 8 and 20, limits inclusive;
p represents an integer between 1 and 10, limits inclusive;
it being understood that at least one of the groups R¹, R² or R⁴ represents a group —(CH₂)ₚ—S—C(XY)—(CH₂)ₙ—C(O)OR³, in which R³, X, Y, n and p are as defined above, said process comprising at least steps a) to c) below:
a) reacting a mercapto alkoxide of formula (2): 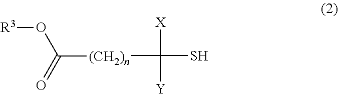
in which R³, X, Y and n are as defined previously, with an amine compound of formula (3): 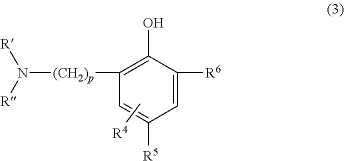
in which R⁴ and p are as defined previously and
R′ and R″, which may be identical or different, are chosen, independently of each other, from a hydrogen atom, a linear or branched alkyl radical comprising from 1 to 6 carbon atoms, or form, together with the nitrogen atom that bears them, a heterocycle,
R⁵ represents a linear or branched hydrocarbon-based group comprising from 1 to 20 carbon atoms or a group —(CH₂)ₚ—NR′R″, and
R⁶ is chosen from a hydrogen atom, a linear or branched hydrocarbon-based group comprising from 1 to 20 carbon atoms, and a group —(CH₂)ₚ—NR′/R″,
b) optionally, reacting the compound obtained in step a) with a compound of formula (4): 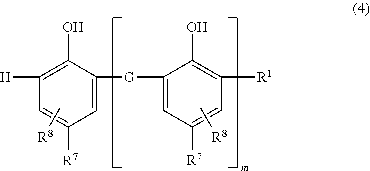
in which R′, R⁷, R⁸, m and G are as defined previously, via an aromatic electrophilic substitution reaction, and
c) extracting and then optionally purifying the compound of formula (1).

2. The process according to claim 1, wherein the radicals X and Y of the compound of formula (1) each represent a hydrogen atom.

3. The process according to claim 1, wherein the radical R⁴ of the compound of formula (1) represents a hydrogen atom.

4. The process according to claim 1, wherein the compound of formula (1) has at least one of the following characteristics:
R¹ is chosen from a hydrogen atom and a linear or branched hydrocarbon-based group comprising from 1 to 6 carbon atoms;
R² represents a group —(CH₂)ₚ—S—C(XY)—(CH₂)ₙ—C(O)OR³;
R³ represents methyl or ethyl;
R⁴ represents a hydrogen atom;
T is chosen from a single bond, —Sᵥ—, —(CH₂)ₜ— and —C(CH₃)₂—;
X represents a radical chosen from a hydrogen atom and methyl, ethyl, propyl and butyl radicals;
Y represents a hydrogen atom;
t represents an integer between 1 and 3, limits inclusive;
v represents an integer between 1 and 6, limits inclusive;
m represents 0, or an integer between 1 and 6, limits inclusive;
n represents an integer between 8 and 12, limits inclusive; and
p is equal to 1.

5. The process according to claim 1, wherein the compound of formula (4) is selected from the group consisting of phenol/aldehyde resins, sulfureous phenolic resins and phenolic resins with gem-dimethyl bridges derived from the oligomerization reaction of ortho-isopropenyl-para-alkylphenol.

6. The process according to claim 1, comprising at least the following steps:
a1) transesterifyinq at least one triglyceride, in the presence of an alcohol, and removing the glycerol formed, to obtain an unsaturated ester;
a1′) optionally, treating said unsaturated ester by metathesis or pyrolysis;
a2) sulhydratinq the unsaturated ester from step a1) or a1′) to obtain the mercapto ester of formula (2) as defined previously;
a) reacting the mercapto ester of formula (2) with an amine phenolic compound of formula (3);
b) optionally, reacting the compound obtained in step a) with a compound of formula (4), optionally in the presence of a reagent bearing the group T;
c) extracting and then optionally purifying the compounds obtained in step b).

7. The process according to claim 1, wherein the raw material of renewable origin is a triglyceride originating from animal or plant oils or fats, selected from the group consisting of soybean oil, sunflower oil, linseed oil, rapeseed oil, castor oil, palm oil, palm kernel oil, coconut oil, jatropha oil, cotton seed oil, groundnut oil, olive oil, vernonia oil, cuphea oil, hevea oil, lunaria oil, safflower oil, camellina oil, Calophyllum inophyllum oil, Pongamia pinnata oil, beef tallow, and cooking oil and grease.

8. A compound of formula (1″):
in which: 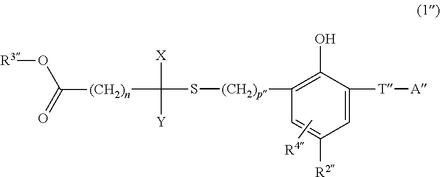
A″ represents a radical R¹″ or a radical of formula A^{m}″: 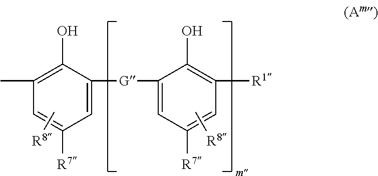
R¹″ is chosen from a hydrogen atom, a linear or branched hydrocarbon-based group comprising from 1 to 20 carbon atoms and a group —(CH₂)_{p″}—S—C(X″Y″)—(CH₂)_{n″}—C(O)OR³″;
R²″ is chosen from a linear or branched hydrocarbon-based group comprising from 1 to 20 carbon atoms and a group —(CH₂)_{p″}—S—C(X″Y″)—(CH₂)_{n″}—C(O)OR³″;
R³″ represents a linear or branched hydrocarbon-based group comprising from 1 to 20 carbon atoms;
R⁴″, R⁷″ and R⁸″, which may be identical or different, are chosen, independently of each other, from a hydrogen atom, a linear or branched hydrocarbon-based group comprising from 1 to 20 carbon atoms and a group —(CH₂)_{p″}—S—C(X″Y″)—(CH₂)_{n″}—C(O)OR³″;
G″ is chosen from —S_{c″}—, —(CH₂)_{a″}—, —C(CH₃)₂—, —[S(O)_{b″}]_{c″}— and —W″—, in which W″ is an aromatic group, optionally substituted with one or more alkyl groups;
T″ is chosen from a single bond, —S_{v″}—, —(CH₂)_{t″}—, —C(CH₃)₂— and —[S(O)_{u″}]_{v″}—;
X″ and Y″, independently of each other, each represent a radical chosen from a hydrogen atom and linear or branched hydrocarbon-based groups, comprising from 1 to 20 carbon atoms, and optionally comprising one or more heteroatoms chosen from oxygen, nitrogen and sulfur;
a″ and t″, which may be identical or different and independently of each other, each represent an integer between 1 and 9 and preferably between 1 and 3, limits inclusive;
b″ and u″, which may be identical or different and independently of each other, each represent an integer equal to 1 or 2;
c″ and v″, which may be identical or different and independently of each other, each represent an integer between 1 and 6, limits inclusive;
m″ represents 0, or an integer between 1 and 20, limits inclusive;
n″ represents an integer between 8 and 20, limits inclusive;
p″ represents an integer between 1 and 10, limits inclusive;
subject to the proviso that at least one of the groups R¹″, R²″ or R⁴″ represents a group —(CH₂)_{p″}—S—C(X″Y″)—(CH₂)ₙ—C(O)OR³″, in which R³″, X″, Y″, n″ and p″ are as defined above, and
subject to the proviso that when p″=1, then A″ represents a radical of formula A^{m}″.

9. The compound according to claim 8, wherein the radicals X″ and Y″ each represent a hydrogen atom.

10. The compound according to claim 8, wherein the radical R⁴″ represents a hydrogen atom.

11. The compound according to claim 8, having at least one of the following characteristics:
R¹″ is selected from a hydrogen atom, a linear or branched hydrocarbon-based group comprising from 1 to 12 carbon atoms or a group —(CH₂)_{p″}13 S—C(X″Y″)—(CH₂)_{n″}—C(O)OR³″;
R²″ is selected from a linear or branched hydrocarbon-based group comprising from 1 to 12 carbon atoms or a group —(CH₂)ₚ—S—C(X″Y″)—(CH₂)_{n″}—C(O)OR³″;
R³″ represents a linear or branched hydrocarbon-based group comprising from 1 to 6 carbon atoms;
R⁴″ is selected from a hydrogen atom or a linear or branched hydrocarbon-based group comprising from 1 to 6 carbon atoms;
T″ is selected from a single bond, —S_{v″}—, —(CH₂)_{t″}— or —C(CH₃)₂—;
X″ and Y″, independently of each other, each represent a radical selected from a hydrogen atom or linear or branched alkyl radicals comprising from 1 to 6 carbon atoms;
t″ represents an integer between 1 and 3, limits inclusive;
v″ represents an integer between 1 and 6, limits inclusive;
m″ represents 0, or an integer between 1 and 10, limits inclusive;
n″ represents an integer between 8 and 20, limits inclusive;
p″ represents 2;
subject to the proviso that at least one of the groups R¹″ or R²″ represents a group —(CH₂)_{p″}—S—C(X″Y″)—(CH₂)_{n″}—C(O)OR³″, in which R³″, X″, Y″, n″ and p″ are as defined above.

12. The compound according to claim 8, having at least one of the following characteristics:
R¹″ is selected from a hydrogen atom or a linear or branched hydrocarbon-based group comprising from 1 to 6 carbon atoms;
R²″ represents a group —(CH₂)_{p″}—S—C(X″Y″)—(CH₂)_{n″}—C(O)OR³″;
R³″ represents methyl or ethyl;
R⁴″ represents a hydrogen atom;
T″ is selected from a single bond, —S_{v″}—, —(CH₂)_{t″}— or —C(CH₃)₂—;
X″ represents a radical selected from a hydrogen atom or methyl, ethyl, propyl or butyl radicals;
Y″ represents a hydrogen atom;
t″ represents an integer between 1 and 3, limits inclusive;
v″ represents an integer between 1 and 6, limits inclusive;
m″ represents 0, or an integer between 1 and 6, limits inclusive;
n″ represents an integer between 8 and 12, limits inclusive; and
p″ is equal to 1.

13. A method, comprising using at least one sulfureous phenolic compound of formula (1″) according to claim 8 as an antioxidant, UV stabilizer or heat stabilizer.

14. The method according to claim 13, wherein the method prepares a product selected from the group consisting of plastics, synthetic fibres, elastomers, adhesives, additives and lubricants.
